(19) United States
(12) Patent Application Publication
(10) Pub. No.: US 2025/0134446 A1
(43) Pub. Date: May 1, 2025

(54) Device of Exciting and Locating Two-photon Fluorescence Signals of Methylene Blue under Skin Surface

(71) Applicant: National Taiwan University, Taipei (TW)

(72) Inventors: Chi-Kuang Sun, Taipei (TW); Pei-Jhe Wu, Taipei (TW); Wei-Zen Sun, Taipei (TW); Chen-Tung Yen, Taipei (TW); Wen-Ying Lin, Taipei (TW)

(21) Appl. No.: 18/541,051

(22) Filed: Dec. 15, 2023

(30) Foreign Application Priority Data
Oct. 30, 2023 (TW) 112141553

(51) Int. Cl. A61B 5/00; G02B 21/16

(52) U.S. Cl. CPC A61B 5/4029; A61B 5/0071; A61B 5/443; G02B 21/16

(57) ABSTRACT

A device is provided to excite and locate methylene blue two-photon fluorescence (MB-2 PF) signals under skin surface. The device comprises a laser source, a nonlinear optical microscope unit, a beam-splitter unit, an optical-signal capturing and observing unit, and an image processing unit. Second harmonic generation (SHG) signals and the MB-2 PF signals are combined and observed simultaneously to form images of nerve fiber structures at different depths under the surface of human skin. The MB-2 PF signals directly observe the nerve fiber structure under the skin surface. The SHG signals reflects the signals of collagen fibers in dermal layer of the skin. The junction between the dermal layer and the epidermal layer are located to divide the dermis and epidermis layers for determining whether the nerve fiber structure passes through the epidermis and dermis layers. Thus, the density of intraepidermal nerve fiber is calculated in a non-invasive way.

### TECHNICAL FIELD OF THE INVENTION

The present invention relates to locating methylene blue (MB) under skin surface; more particularly, to imaging by simultaneously exciting and locating fluorescence signals of MB under the skin surface in a non-invasive way, where the structural image of neural fibers are observed as a basis for real-time related clinical diagnosis for the future.

### DESCRIPTION OF THE RELATED ARTS

For now, skin nerve biopsy is the only standard medical method for observing a neural structure and is an invasive one, which requires the use of special chemical immunostaining. This invasive method mainly removes skin tissue from body with a live piercing puncture device under local anesthesia, where it takes 7 to 10 days to heal the wound. Then, after using a special chemical immunostaining, the neural structure and the density of intraepidermal nerve fiber are observed under a microscope, where it takes 1 month to obtain a pathological report. As described above, the invasive method is the current way of observing the nerves in hospital neurology, yet the required excision of tissue causes pain and pathological staining is complicated and time-consuming.

For observing nerve fibers in human skin by using MB two-photon fluorescence (2 PF) signals alone, a solution with a low MB concentration is injected into human body, which will be metabolized from the body over time, especially from terminal nerves; that is, the 2 PF signals of the terminal nerves in epidermis will begin to degrade and cannot be used for long-term observation. Therefore, third harmonic generation (THG) signals are combined in conventional techniques for compensation due to the problem of the gradual metabolization of the MB solution from the body over time. However, due to the clinical need of a clear definition in determining the number of nerves in epidermal as the current criterion for clinical diagnosis of peripheral neuropathy, the MB-2 PF signals are applied with the THG signals for observing the dermal and epidermal nerve signals in the skin. Nevertheless, it is still not possible to determine the location of the nerve signals in the skin space and, hence, cannot effectively quantify the intraepidermal nerve fiber density in the skin. Hence, the prior arts do not fulfill all users' requests on actual use.

### SUMMARY OF THE INVENTION

The main purpose of the present invention is to combine second harmonic generation (SHG) signals and the MB-2 PF signals for directly observing signals of nerve fibers under human skin, where the SHG signals are used to observe whether the nerve fibers pass through the junction between the dermal layer and the epidermal layer for further defining the density of terminal nerves.

To achieve the above purpose, the present invention is a device of exciting and locating MB-2 PF signals under skin surface, comprising a laser unit, a nonlinear optical microscope unit, a beam-splitter unit, an optical-signal capturing and observing unit, and an image processing unit, where the laser unit emits a laser beam of pulsed laser with a specific infrared band; the nonlinear optical microscope unit has an objective and projects a laser beam into a human skin treated with a low-density MB dye to capture and observe reverse light signals excited and generated in the human skin through the laser beam; the reverse light signals comprises SHG light and MB-2 PF light; the beam-splitter unit splits the reverse light signals into the SHG light and the MB-2 PF light; the optical-signal capturing and observing unit captures and observes the SHG light and the MB-2 PF light, respectively, to convert the SHG light and the MB-2 PF light into corresponding SHG signals and MB-2 PF signals; the image processing unit combines and simultaneously observes the SHG signals and the MB-2 PF signals to obtain images of nerve fiber structure at different depths under the surface of the human skin; the MB-2 PF signals directly obtains the nerve fiber structure under the surface of the human skin and the SHG signals reflects the signals of collagen fiber in dermal layer of the human skin; and epidermis layer and dermis layer are distinguished to locate a junction between the epidermis layer and the dermis layer to find the places where the nerve fiber structure passes through the epidermis and dermis layers to calculate the density of intraepidermal nerve fiber in a non-invasive way. Accordingly, a novel device of exciting and locating MB-2 PF signals under skin surface is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the structural view showing the preferred embodiment according to the present invention; and

FIG. 2 is the view showing the photograph of the second harmonic generation (SHG) signals and the methylene blue (MB) two-photon fluorescence (2 PF) signals.

### DESCRIPTION OF THE PREFERRED EMBODIMENT

The following description of the preferred embodiment is provided to understand the features and the structures of the present invention.

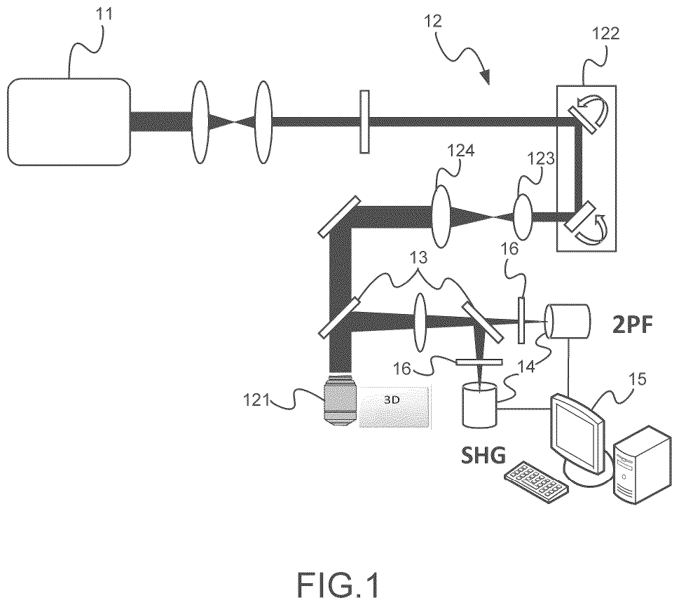

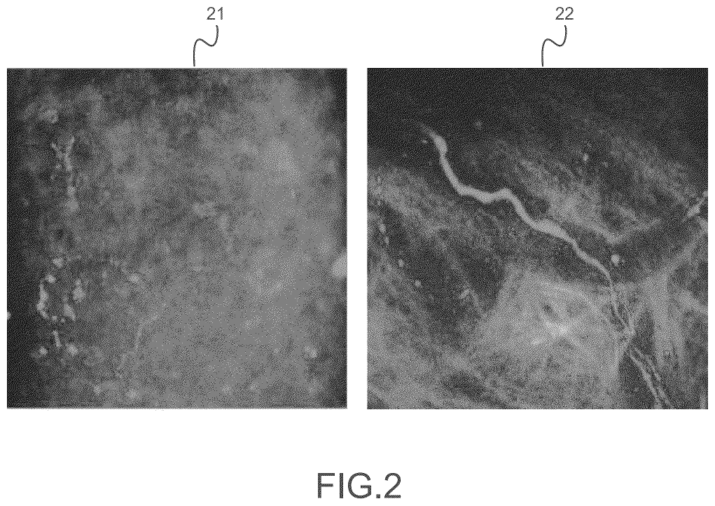

Please refer to FIG. 1 and FIG. 2, which are a structural view showing a preferred embodiment according to the present invention; and a view showing a photograph of SHG signals and MB-2 PF signals. As shown in the figures, the present invention is a device of exciting and locating MB-2 PF signals under skin surface, comprising a laser unit 11, a nonlinear optical microscope unit 12, a beam-splitter unit 13, an optical-signal capturing and observing unit 14, and a image processing unit 15.

The laser unit 11 emits a laser beam of pulsed laser with a specific infrared (IR) band, where the laser unit 11 is a laser source having a wavelength between 1100 to 1450 nanometer (nm) and a laser pulse width smaller than 10 picoseconds (ps).

The nonlinear optical microscope unit 12 has an objective 121. The objective 121 is a microscope objective lens with a numerical aperture (NA) not smaller than 0.75, that projects the laser beam into a human skin treated with a low density of a dye of MB to capture and observe reverse light signals excited and generated in the human skin through the laser beam. Therein, the reverse light signals comprises SHG light and MB-2 PF light.

The beam-splitter unit 13 is a dichroic beam splitter (DBS), where the reverse light signals are split into the SHG light and the MB-2 PF light.

The optical-signal capturing and observing unit 14 is a photomultiplier (PMT), where the SHG light and the MB-2 PF light are captured and observed to be converted into corresponding SHG signals and MB-2 PF signals, respectively.

The image processing unit 15 combines and simultaneously observes the SHG signals and the MB-2 PF signals to form images of nerve fiber structure at different depths under the surface of the human skin, where the MB-2 PF signals are used to observe the nerve fiber structure under the surface of the human skin and the SHG signals reflects the signals of collagen fiber in dermal layer of the human skin; and epidermis layer and dermis layer are distinguished to locate a junction between the epidermis layer and the dermis layer for finding the places where the nerve fiber structure passes through the epidermis and dermis layers for calculating the density of intraepidermal nerve fiber in a non-invasive way. Thus, a novel device of exciting and locating MB-2 PF signals under skin surface is obtained.

On using the present invention, with a low-density MB dye clinically injected into the human skin, a nonlinear optical microscope device is provided to simultaneously excite MB and observe its 2 PF signal for obtaining non-invasive observational neural images. With SHG signals of collagen fibers, the MB signals in skin are located for quantitative parameter analysis (e.g. density of intraepidermal nerve fiber). Thus, the same effect as conventional pathological analysis is achieved without the need of time-consuming chemical immunostaining, where the basis of timely clinical diagnosis for the future is provided for physicians.

In a state-of-use, the nonlinear optical microscope unit 12 has an optical high-frequency galvanometer scanner 122. A high-frequency scan lens 123 and a tube lens 124 are set between the optical high-frequency galvanometer scanner 122 and the objective 121. After a laser beam is reflected by the optical high-frequency galvanometer scanner 122 and is expanded by the high-frequency scan lens 123 and the tube lens 124, a point is formed at the focus of the objective 121 to excite and generate reverse light signals.

In a state-of-use, a bandpass filter (BPF) 16 is located between the beam-splitter unit 13 and the optical-signal capturing and observing unit 14 to filter out unwanted frequencies in the SHG light and the MB-2 PF light obtained from the beam-splitter unit 13 and entered into the optical-signal capturing and observing unit 14 after being filtered.

In a state-of-use, an assessing and judging unit (not shown in the figure) is further comprised to assess the damage degree of the terminal nerves of the human skin to determine related neurological disorder, such as peripheral neuropathy. In the other words, the more severely is the terminal nerves damaged, the lower is the density of intraepidermal nerve fiber.

Given the definition of intraepidermal nerve fiber density, it is crucial to know how many of the terminal nerves enter into the epidermis layer through the dermal layer, which affects the number of the terminal nerves calculated within a unit of area. Hence, the present invention combines MB-2 PF signals and SHG signals to be applied for nerve fibers of human skin, which aims at addressing and overcoming deficiencies in past related clinical treatments. The SHG signals are used to observe collagen fiber signals in the dermis layer of the human skin. Because the collagen fiber signals are located in the dermis layer only, this phenomenon can be applied to further confirm the locations of the MB-2 PF signals in the three-dimensional space of the human skin, which can be applied clinically for the future to non-invasively quantify the density of intraepidermal nerve fiber.

As shown in FIG. 2, the present invention combines SHG signals 21 and MB-2 PF signals 22 obtained in a non-invasive way for directly observing the signals of nerve fibers under a human skin. Because the SHG signals are observed in the collagen fibers of dermis layer (for no collagen fiber in epidermal layer), they are used to determine whether the nerve fibers pass through the junction between the dermis layer and the epidermal layer for further defining the density of intraepidermal nerve fiber.

To sum up, the present invention is a device of exciting and locating MB-2 PF signals under skin surface, where a non-invasive imaging way is used to simultaneously excite and locate MB-2 PF signals under the skin surface for observing the structure of neural fibers as a basis for real-time related clinical diagnosis for the future.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A device of exciting and locating MB-2 PF signals under skin surface, comprising
a laser unit,
wherein said laser unit emits a laser beam of pulsed laser with a specific infrared (IR) band;
a nonlinear optical microscope unit,
wherein said nonlinear optical microscope unit has an objective and projects a laser beam into a human skin treated with a low-density MB dye to capture and observe reverse light signals excited and generated in the human skin through the laser beam, said reverse light signals comprising second harmonic generation (SHG) light and MB two-photon fluorescence (2 PF) light;
a beam-splitter unit,
wherein said beam-splitter unit splits said reverse light signals into said SHG light and said MB-2 PF light;
an optical-signal capturing and observing unit,
wherein said optical-signal capturing and observing unit captures and observes said SHG light and said MB-2 PF light, respectively, to convert said SHG light and said MB 2 PF light into corresponding SHG signals and MB-2 PF signals; and
an image processing unit,
wherein said image processing unit combines and simultaneously observes said SHG signals and said MB-2 PF signals to obtain images of nerve fiber structure at different depths under the surface of said human skin;
wherein said MB-2 PF signals directly obtains said nerve fiber structure under the surface of said human skin and said SHG signals reflects the signals of collagen fiber in dermal layer of said human skin; and
wherein epidermis layer and dermis layer are distinguished to locate a junction between said epidermis layer and said dermis layer to find the places where said nerve fiber structure passes through said epidermis and dermis layers to calculate the density of intraepidermal nerve fiber in a non-invasive way.

2. The device according to claim 1,
wherein said laser unit is a laser source using a wavelength between 1100 to 1450 nanometer (nm) and a laser pulse width smaller than 10 picoseconds (ps).

3. The device according to claim 1,
wherein said objective is a microscope objective lens with a numerical aperture (NA) not smaller than 0.75.

4. The device according to claim 1,
wherein said beam-splitter unit is a dichroic beam splitter (DBS).

5. The device according to claim 1,
wherein said nonlinear optical microscope unit has an optical high-frequency galvanometer scanner; a high-frequency scan lens and a tube lens are located between said optical high-frequency galvanometer scanner and said objective; and, after said laser beam is reflected by said optical high-frequency galvanometer scanner and is expanded by said high-frequency scan lens and said tube lens, a point is obtained at the focus of said objective to excite and generate said reverse light signals.

6. The device according to claim 1,
wherein said optical-signal capturing and observing unit is a photomultiplier (PMT).

7. The device according to claim 1,
wherein a bandpass filter (BPF) is located between said beam-splitter unit and said optical-signal capturing and observing unit to filter out unwanted frequencies in said SHG light and said MB-2 PF light obtained from said beam-splitter unit and entered into said optical-signal capturing and observing unit after being filtered.
